(19) United States
(12) Patent Application Publication
(10) Pub. No.: US 2004/0213821 A1
(43) Pub. Date: Oct. 28, 2004

(54) Skin care composition comprising first and second emulsions

(75) Inventors: Yasuko Suginaka, Ashiya (JP); Hidekazu Tanaka, Kobe (JP)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/769,278

(22) Filed: Jan. 30, 2004

Related U.S. Application Data
(60) Provisional application No. 60/444,273, filed on Jan. 31, 2003.

(51) Int. Cl. A61K 6/00; A61K 7/00

(52) U.S. Cl. 424/401

(57) ABSTRACT

Disclosed is a skin care composition comprising:

(a) a first emulsion comprising: a first surfactant system comprising nonionic surfactants; a first oily component is selected from the group consisting of emollients, silicone oils, and mixtures thereof; and a first aqueous carrier; wherein the weight ratio of the first surfactant system to the first oily component is from about 5:1 to about 1:1;

(b) a second emulsion comprising: a second surfactant system comprising nonionic surfactants; a second oily component is selected from the group consisting of perfumes, hydrophobic plant extracts containing terpene compounds, hydrophobic vitamins, and mixtures thereof; and a second aqueous carrier; wherein the second oily component comprises and wherein the weight ratio of the second surfactant system to the second oily component is from about 20:1 to about 3:1; and

(c) a third aqueous carrier.

### CROSS REFERENCE TO RELATED APPLICATION

This application claims the benefit of U.S. Provisional application No. 60/444,273, filed on Jan. 31, 2003.

### FIELD OF INVENTION

The present invention relates to a skin care composition comprising a first emulsion and a second emulsion. Especially, the present invention relates to transparent or translucent skin care composition providing aesthetic benefits and/or additional conditioning benefits, while not deteriorating transparency and skin conditioning benefits such as moisturization, smoothness and softness.

### BACKGROUND OF THE INVENTION

Many personal care products currently available to consumers are directed primarily to improving the health and/or physical appearance of the skin. Among these skin care products, many are directed to delaying, minimizing or even eliminating skin wrinkling and other histological changes typically associated with skin aging or environmental damage to human skin.

In order to maintain or return skin to a healthy and/or youthful state, the skin is typically treated with a moisturizing agent. Known moisturizing agents include, for example, water soluble humectants such as glycerin. Addition of oily components is also known to be advantageous, in that they provide softness, smoothness, and supplement of intercellular lipid.

Transparent forms of lotions and emulsions incorporating water soluble humectants and oils, to provide moisturization, smoothness and softness to the skin, are known in the art such as PCT publications WO99/49841, WO00/37029, WO00/61098, and WO00/61083. The transparent appearance is appealing to consumers, as there is a belief that application of such compositions will make their skin transparent and young-looking. It would be even more appealing to consumers if such products provide other benefits such as aesthetic benefits and/or additional conditioning benefits, in addition to moisturization, smoothness and softness. Such aesthetic benefits and/or additional conditioning benefits can be obtained from components such as perfumes, hydrophobic plant extracts, and hydrophobic vitamins.

It is desired to incorporate materials such as perfumes, hydrophobic plant extracts, and hydrophobic vitamins, into the transparent forms of lotions and emulsions, without deteriorating transparency, moisturization, smoothness, and softness of the composition.

Based on the foregoing, there exists a need for transparent or translucent skin care composition providing aesthetic benefits and/or additional conditioning benefits, while not deteriorating transparency and skin conditioning benefits such as moisturization, smoothness and softness.

None of the existing art provides all of the advantages and benefits of the present invention.

### SUMMARY OF THE INVENTION

The present invention is directed to a skin care composition comprising:

(a) a first emulsion comprising a first surfactant system, a first oily component, and a first aqueous carrier; wherein the first surfactant system comprises two or more nonionic surfactants selected from the group consisting of polyoxyalkylene alkyl ethers having the C12-18 of alkyl substitute, polyoxyalkylene hydrogenated caster oils, and a linear or branched, mono-or tri-alkyl glycerides; wherein the first oily component is selected from the group consisting of emollients, silicone oils, and mixtures thereof; and wherein the weight ratio of the first surfactant system to the first oily component is from about 5:1 to about 1:1;

(b) a second emulsion comprising a second surfactant system, a second oily component, and a second aqueous carrier; wherein the second surfactant system comprises one or more nonionic surfactants selected from the group consisting of polyoxyalkylene hydrogenated caster oils, polyglycerin alkyl esters having the C10-20 of alkylsubstitute, polysorbates, polyoxyethylene sterols, polyoxyethylene hydrogenated sterols, hydroxylated lecithin, polyoxyethylene alkyl ether phosphates and salts thereof, and glycereth-25 PCA isostearate; wherein the second oily component comprises is selected from the group consisting of perfumes, hydrophobic plant extracts containing terpene compounds, hydrophobic vitamins, and mixtures thereof; and wherein the weight ratio of the second surfactant system to the second oily component is from about 20:1 to about 3:1; and

(c) a third aqueous carrier.

These and other features, aspects, and advantages of the present invention will become better understood from a reading of the following description, and appended claims.

All documents cited are, in relevant part, incorporated herein by reference; the citation of any document is not to be construed as an admission that it is prior art with respect to the present invention.

### DETAILED DESCRIPTION OF THE INVENTION

While the specification concludes with claims particularly pointing out and distinctly claiming the invention, it is believed that the present invention will be better understood from the following description.

Herein, “comprising” means that other steps and other ingredients which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Herein, “mixtures” is meant to include a simple combination of materials and any compounds that may result from their combination. While the specification concludes with claims which particularly point out and distinctly claim the invention, it is believed the present invention will be better understood from the following description.

Herein, “comprising” means that other steps and other ingredients which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Composition

The composition of the present invention is suitable for topical use on human body skin, particularly suitable for facial skin. The composition has a transparent to translucent appearance, and thus is particularly suitable for a clear lotion product. Typically, applications would be on the order of about once per day over such extended periods, while application rates can be up to about three times per day or more.

The present compositions can provide aesthetic benefits and/or additional conditioning benefits, while not deteriorating transparency and skin conditioning benefits such as moisturization, smoothness and softness.

The “base composition” of the present composition has high transparency. By “base composition”, what is meant is the composition of the present invention containing only the essential components, i.e., a composition consisting essentially of the first emulsion, the second emulsion, and the third aqueous carrier. The base composition has an absorbance of less than about 2 at a wave length of 340 nm; preferably from about 1 to about 1.5 at the same wave length. Absorbance herein can be determined by using the Micro Plate Reader by TECAN Co. Ltd.

First Emulsion

The composition of the present invention comprises a first emulsion. The first emulsion comprises a first surfactant system, a first oily component, and a first aqueous carrier. The first emulsion may further contain preservatives such as benzyl alcohol.

Preferably, the first emulsion have a mean particle size of less than about 100 nm, more preferably less than about 80 nm. The size of the droplets can be determined using a Laser Scattering Particle Size Distribution Analyzer LA-910 by Horiba (Japan). The size of droplets is measured with the emulsion at a temperature of 25° C.+/−1° C., after 30 seconds mixing. The first emulsion has a form of a microemulsion. Herein, “microemulsion” means an emulsion containing an aqueous carrier as the continuous phase, in which water-insoluble components, which would otherwise provide a non-transparent appearance, are dispersed as such small droplets that they appear as a transparent emulsion. One skilled in the art understands certain oily components may appear transparent even when dispersed at a larger droplet size. In the present invention, the droplet size of the oily components that do not appear transparent at larger droplet size, are critical for providing the base composition transparent. Without being bound by theory, it is believed the selection of species and amount of the surfactant system provides a suitable microemulsion for dispersing the oily components. The weight ratio of the first surfactant system to the first oily component is from about 5:1 to about 1:1.

First Surfactant System

The first surfactant system comprises two or more nonionic surfactants selected from the group consisting of polyoxyalkylene alkyl ethers having the C 12-18 of alkyl substitute, polyoxyalkylene hydrogenated caster oils, and a linear or branched, mono-or tri-alkyl glycerides.

Polyoxyalkylene alkyl ethers useful herein are the condensation products of alkylene oxides with both fatty acids and fatty alcohols (e.g., wherein the polyalkylene oxide portion is esterified on one end with a fatty acid and etherified (e.g., connected via an ether linkage) on the other end with a fatty alcohol). These materials have the general formula R¹CO(X₁)zOR² wherein R¹ and R² are independently alkyl of from about 12 to about 18 carbons; X₁ is —OCH₂CH₂ derived from, for example ethylene glycol or —OCH₂CHCH₃— derived from propylene glycol or oxide; and z is an integer from about 6 to about 50.

Nonlimiting examples of such alkylene oxide derived nonionic surfactants include ceteth-6, ceteth-10, ceteth-12, ceteareth-6, ceteareth-10, ceteareth-12, ceteareth-20, ceteareth-30, steareth-6, steareth-10, steareth-12, steareth-20, PEG-100 steareth, PEG-6 stearate, PEG-10 stearate, PEG-12 stearate, PEG-100 stearate, PEG-10 glyceryl stearate, PEG-20 glyceryl stearate, PEG-30 glyceryl cocoate, PEG-80 glyceryl cocoate, PEG-80 glyceryl tallowate, PEG-200 glyceryl tallowate, PEG-8 dilaurate, PEG-10 distearate, and mixtures thereof; preferably, ceteareth-12, ceteareth-20, and ceteareth-30. Commercially available surfactants include ceteareth-12 with tradename Eumulgin B1, ceteareth-20 with tradename Eumulgin B2, and ceteareth-30 with tradename Eumulgin B3, all available from Henkel.

Examples of polyoxyalkylene hydrogenated castor oil useful herein include polyoxyethylene hydrogenated caster oil; preferably polyoxyethylene (20) hydrogenated caster oil.

Examples of mono- or tri-alkyl glyceride useful herein include glyceryl monostearate, glyceryl oleate, and triglyceryl diisostearate; preferably triglyceryl diisostearate.

The first surfactant system is contained in the composition of the present invention at a level of preferably from about 0.02% to about 3%, more preferably from about 0.1% to about 2% by weight of the composition.

The hydrophilic-lipophilic balance (HLB) of the first surfactant system is selected by the artisan in view of the amount and species of the first oily component. Preferably, the HLB of the first surfactant system as a whole is higher than about 10. Preferably, among the surfactants used for the first surfactant system, at least one surfactant has an HLB of more than 10, and at least one surfactant has an HLB of below 10. More preferably, the amount of higher HLB surfactants is greater than the lower HLB surfactants. Without being bound by theory, by using both a higher HLB surfactant and a lower HLB surfactant, a stable emulsion can be made with as low level of surfactant possible. In a particularly preferred embodiment, at least 3 surfactants are used, such surfactants each having different HLBs.

In a preferred method of preparing the composition of the present invention, the first surfactant system is first mixed with the first oily component, and such mixture is added to the first aqueous carrier. The amount of the first aqueous carrier used in this step is preferably from about 0.5 to about 10 times, more preferably from about 1 to about 7 times the total weight of the first oily component and the first surfactant system. Without being bound by theory, by incorporating the surfactant system in such manner, a stable emulsion can be made with as low level of surfactant possible.

First Oily Component

The first oily components comprise one or more selected from the group consisting of (i) emollients, (ii) silicone oils, and mixtures thereof. The first oily components useful herein provides moisturizing efficacy to the skin.

The first oily component is preferably heat tolerant at 80° C. By “oily component being heat tolerant at 80“C”, what is meant is that the oily component meets following conditions at 80° C., preferably 82° C., more preferably 85° C.: (i) the oily component is not volatile; (ii) the oily component is not irreversibly thermally denatured.

The first oily component, which is encompassed in the first emulsion, can be contained in the composition at a level of preferably from about 0.02% to about 2%, more preferably from about 0.05% to about 2% by weight of the composition.

(i) Emollient

The emollient oil useful herein are those having a melting point of not more than about 25° C., and provide emollient benefit to the skin. The emollient oils useful herein include esters and hydrocarbons. Emollient oils of lower viscosity, low molecular weight, or branched structure, are highly preferable. It has been surprisingly found that, by the use of such oils, the tacky and greasy feel to the skin can be alleviated.

Emollient oils useful herein are esters, particularly esters having branched alkyl and alkenyl groups. Such esters include, for example, cetyl 2-ethylhexanoate, tridecyl isononanoate, isostearyl isostearate, isocetyl isosteatrate, isopropyl isostearate, isodecyl isonoanoate, cetyl octanoate, isononyl isononanoate, diisopropyl myristate, isocetyl myristate, isotridecyl myristate, isopropyl myristate, myristyl myristate, isostearyl palmitate, isocetyl palmitate, isodecyl palmitate, isopropyl palmitate, isostearyl myristate, octyl palmitate, caprylic/capric acid triglyceride, glyceryl tri-2-ethylhexanoate, neopentyl glycol di(2-ethyl hexanoate), neopentyl glycol dicaprate, diisopropyl dimerate, glycerol trioctanate, glycerol triisopalmitate, isopropyl myristate, octyldodecyl lactate, and mixtures thereof. Triglycerides such as caprylic/capric triglyceride, PEG-6 caprylic/capric triglyceride, and PEG-8 caprylic/capric triglyceride may also be useful. Crude mixtures of such triglycerides by the CTFA name Meadowfoam seed oil are also useful. Commercially available oils include, for example, isononyl isononanoate with tradenames Salacos 99 available from Nisshin Oil Mills, or Lanol 99 available from Seppic; tridecyl isononanoate with tradename Crodamol TN available from Croda, and Hexalan available from Nisshin Seiyu, and Meadowfoam Seed Oil with tradename Cropure MDF available from Croda.

Emollient oils also useful herein are the various grades and types of hydrocarbons. Mineral oils are liquid mixtures of hydrocarbons that are obtained from petroleum. Specific examples of suitable hydrocarbons include paraffin oil including those called light paraffin or isoparaffin, mineral oil, squalane, dodecane, isododecane, hexadecane, isohexadecane, eicosene, isoeicosene, tridecane, tetradecane, hydrogenated polyisobutylene, docosane, and mixtures thereof. Commercially available hydrocarbons useful herein include isododecane, isohexadeance, and isoeicosene with tradenames PERMETHYL 99A, PERMETHYL 10A, and PERMETHYL 1082, available from Presperse (South Plainfield New Jersey, USA), mineral oil with tradename BENOL available from Witco, isoparaffin with tradename ISOPAR available from Exxon Chemical Co. (Houston Tex., USA.), tradename Isoparaffin 2028 available from Idemitsu, and tradename Amsco Mineral Spirits available from Ashland.

(ii) Silicone Oils

The silicone components useful herein include insoluble silicones suitable for use on the skin. By insoluble what is meant is that the silicone forms a separate, discontinuous phase from the carrier, such as in the form of an emulsion or a suspension of droplets of the silicone. The silicone components herein may be made by any suitable method known in the art, including emulsion polymerization. The silicone components may further be incorporated in the present composition in the form of an emulsion, wherein the emulsion is made my mechanical mixing, or in the stage of synthesis through emulsion polymerization, with or without the aid of a surfactant selected from anionic surfactants, nonionic surfactants, cationic surfactants, and mixtures thereof. Silicone components of high molecular weight may be made by emulsion polymerization.

Silicone components useful herein include polyalkyl polyaryl siloxanes, silicone resins, amino-substituted siloxanes, and mixtures thereof. The silicone component is preferably selected from the group consisting of polyalkyl polyaryl siloxanes, silicone resins, and mixtures thereof, and more preferably from one or more polyalkyl polyaryl siloxanes.

Polyalkyl polyaryl siloxanes useful here in include those with the following structure (I)

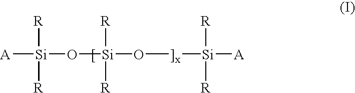

wherein R is alkyl or aryl, and x is an integer from about 7 to about 8,000. “A” represents groups which block the ends of the silicone chains. The alkyl or aryl groups substituted on the siloxane chain (R) or at the ends of the siloxane chains (A) can have any structure as long as the resulting silicone remains fluid at room temperature, is dispersible, is neither irritating, toxic nor otherwise harmful when applied to the skin, is compatible with the other components of the composition, is chemically stable under normal use and storage conditions, and is capable of being deposited on and conditions the skin. Suitable A groups include hydroxy, methyl, methoxy, ethoxy, propoxy, and aryloxy. The two R groups on the silicon atom may represent the same group or different groups. Preferably, the two R groups represent the same group. Suitable R groups include methyl, ethyl, propyl, phenyl, methylphenyl and phenylmethyl. The preferred silicone compounds are polydimethylsiloxane, polydiethylsiloxane, and polymethylphenylsiloxane. Polydimethylsiloxane, which is also known as dimethicone, is especially preferred. The polyalkylsiloxanes that can be used include, for example, polydimethylsiloxanes. These silicone compounds are available, for example, from the General Electric Company in their Viscasil and SF 96 series, and from Dow Corning in their Dow Corning 200 series. Polymethylphenylsiloxanes, for example, from the General Electric Company as SF 1075 methyl phenyl fluid or from Dow Corning as 556 Cosmetic Grade Fluid, are useful herein.

Another polyalkyl polyaryl siloxane that can be especially useful is a silicone gum. The term “silicone gum”, as used herein, means a polyorganosiloxane material having a viscosity at 25° C. of greater than or equal to 1,000,000 centistokes. It is recognized that the silicone gums described herein can also have some overlap with the above-disclosed silicone compounds. This overlap is not intended as a limitation on any of these materials. Silicone gums are described by Petrarch, and others including U.S. Pat. No. 4,152,416, to Spitzer et al., issued May 1, 1979 and Noll, Walter, Chemistry and Technology of Silicones, New York: Academic Press 1968. Also describing silicone gums are General Electric Silicone Rubber Product Data Sheets SE 30, SE 33, SE 54 and SE 76. All of these described references are incorporated herein by reference in their entirety. The “silicone gums” will typically have a mass molecular weight in excess of about 200,000, generally between about 200,000 and about 1,000,000. Specific examples include polydimethylsiloxane, poly(dimethylsiloxane methylvinylsiloxane) copolymer, poly(dimethylsiloxane diphenylsiloxane methylvinylsiloxane) copolymer and mixtures thereof.

The method of manufacturing these silicone components, can be found in Encyclopedia of Polymer Science and Engineering, Volume 15, Second Edition, pp. 204-308, John Wiley & Sons, Inc., 1989.

In one preferred embodiment, the silicone component is a mixture of high viscosity silicone compounds and silicone based carriers. High viscosity silicone compounds herein include those having a molecular weight of from about 200,000 to about 540,000 selected from those mentioned above, preferably selected from the group consisting of dimethiconol, fluorosilicone dimethicone, and mixtures thereof, more preferably essentially dimethiconol. Particularly preferred dimethiconols are those having dimethylpolysiloxane repeating units, and terminated with hydroxy groups, wherein the dimethylsiloxane portion is made of from about 2700 to about 4500 repeating units. Silicone based carriers include those having a viscosity of from about 6 mPa·s to about 100 mPa·s selected from cyclomethicones and dimethicones having lower repeating units.

Commercially available silicone components which are useful herein include Dimethicone with tradename DC345 available from Dow Corning Corporation, Dimethicone gum solutions with tradenames SE 30, SE 33, SE 54 and SE 76 available from General Electric, Dimethiconol with tradenames DCQ2-1403 and DCQ2-1401 available from Dow Corning Corporation, and emulsion polymerized Dimethiconol available from Toshiba Silicone as described in GB application 2,303,857.

Second Emulsion

The composition of the present invention comprises a second emulsion. The second emulsion comprises a second surfactant system, a second oily component, and a second aqueous carrier. Preferably, the second emulsion further contain a polyol carrier. The particle size of the second emulsion can be in a range of preferably about 50 nm or less, more preferably about 30 nm or less.

The weight ratio of the second surfactant system to the second oily component is from about 20:1 to about 3:1, preferably from about 15:1 to 5:1.

Second Surfactant System

The second surfactant system comprises one or more nonionic surfactants selected from the group consisting of polyoxyalkylene hydrogenated caster oils, polyglycerin alkyl esters having the C10-20 of alkylsubstitute, polysorbates, polyoxyethylene sterols, polyoxyethylene hydrogenated sterols, hydroxylated lecithin, polyoxyethylene alkyl ether phosphates and salts thereof, and glycereth-25 PCA isostearate. Preferably, the second surfactant system comprises two or more nonionic surfactants selected from the above species.

Polyoxyalkylene hydrogenated castor oils useful herein include, for example, polyoxyethylene hydrogenated castor oils having 20-100 moles of ethylene oxides, such as polyoxyethylene (20) hydrogenated castor oil, polyethylene (40) hydrogenated castor oil, and polyoxyethylene (100) hydrogenated castor oil.

Polyglycerin alkyl esters having the C10-20 of alkylsubstitute useful herein include, for example, those having 6-10 moles of glycerin units, such as polyglyceryl-6 laurate, polyglyceryl-10 laurate, and polyglyceryl-10 stearate.

Polysorbates useful herein include, for example, those having 20-80 moles of ethylene oxides, such as polysorbate-20, polyborbate-40, polysorbate-60, and polysorbate-80.

Polyethylene sterols and polyethylene hydrogenated sterols useful herein include, for example, those having 10-30 moles of ethylene oxides, such as polyethylene (10) phytosterol, polyethylene (30) phytosterol, and polyethylene (20) cholesterol.

Polyoxyethylene alkyl ether phosphates useful herein include, for example, those having one or two polyoxyethylene alkyl ether groups wherein the alkyl portion has from about 10 to about 20 carbon atoms. Such polyoxyethylene alkyl ether phosphates are preferably included as salts in the composition of the present invention. Salts of the polyoxyethylene alkyl ether phosphates useful herein include, for example, sodium salts, ammonium salts, and potassium salts. Preferred are, for example, sodium dilaureth-10 phosphate, and sodium didioleth-8 phosphate.

The second surfactant system is contained in the composition of the present invention at a level of preferably from about 0.003% to about 3%, more preferably from about 0.01% to about 2% by weight of the composition.

The hydrophilic-lipophilic balance (HLB) of the second surfactant system is selected by the artisan in view of the amount and species of the second oily component._Preferably, the HLB of the second surfactant system as a whole is higher than about 8, more preferably higher than about 10. Preferably, the second surfactant system consists essentially of nonionic surfactants having an HLB of 8 or more, more preferably 10 or more. Without being bound by theory, by using only higher HLB surfactant, the second emulsion can be contained in the present composition without deteriorating its transparency.

In a preferred method of preparing the composition of the present invention, the second surfactant system is first mixed with the second oily component, and such mixture is added to the second aqueous carrier. The amount of the second aqueous carrier used in this step is preferably from about 0.5 to about 20 times, more preferably from about 1 to about 10 times the total weight of the second oily component and the second surfactant system. Without being bound by theory, by incorporating the surfactant system in such manner, a stable emulsion can be made with as low level of surfactant possible.

Second Oily Component

The second oily components comprise one or more selected from the group consisting of perfumes, hydrophobic plant extracts containing terpene compounds, hydrophobic vitamins, and mixtures thereof.

In the present composition, the second oily components are emulsified separately from the first oily component, and preferably emulsified at a temperature of about 20-40° C. Preferably, in the present composition, the second oily components especially perfumes are present in form of only the second emulsion. It is believed that; by incorporating the second oily components into the composition in form of the second emulsion, the composition can have improved stability. It has been found by the inventors that; when incorporating second oily components, especially perfumes, into the composition without the preparation of a second emulsion encompassing the second oily component, the oily components deteriorate the transparency or translucency of the composition, i.e., the composition becomes opaque or milky, especially during the storage at a temperature of 50° C. or above.

Perfumes useful herein include one perfume component and a mixture of two or more perfume components. Such perfume components useful herein include natural perfume components and synthetic perfume components. Synthetic perfumes highly useful herein include, fore example, terpenes such as menthol, camphor, and limonene; and aldehydes such as γ-heptyl butyrolactone and ethyl β-methyl-β-phenyl glycidate.

Hydrophobic plant extracts containing terpene compounds useful herein include, for example, tea tree oil containing terpinene-4-ol and γ-terpinene and chamomile extract containing bisarol.

Hydrophobic vitamins useful herein include, for example, vitamin A and its derivatives such as vitamin A palmitate and vitamin A propyonate, and vitamin D and its derivatives.

The second oily component can be contained in the composition at a level of preferably from about 0.001% to about 1%, more preferably from about 0.01% to about 0.5% by weight of the composition.

Polyol Carrier

Preferably the second emulsion further contains a polyol carrier. Polyol carriers useful herein include, for example, 1,3-butylene glycol, glycerin, pentylene glycol, and propylene glycol. Some of these polyol carriers can be also used as “WATER SOLUBLE HUMECTANT” described below.

The polyol carrier can be contained in the composition at a level of preferably from about 0.01% to about 10%, more preferably from about 0.1% to about 6% by weight of the composition. The weight ratio of the polyol carrier to the second surfactant system is preferably from about 5:1 to about 1:5, more preferably from about 3:1 to about 1:3.

It is believed that, polyol carriers help the second emulsion form, by solving the second surfactant system. In a preferred method of preparing the composition of the present invention, the polyol carrier is first mixed with second surfactant system and the second oily component, and such mixture is added to the second aqueous carrier.

Aqueous Carrier

The compositions of the present invention comprise aqueous carriers. The level and species of the carriers are selected according to the compatibility with other components, and other desired characteristic of the product.

Carriers useful in the present invention include water and water solutions of lower alkyl alcohols. Lower alkyl alcohols useful herein are monohydric alcohols having 1 to 6 carbons, more preferably ethanol and isopropanol.

Preferably, aqueous carriers are substantially water. Deionized water is preferably used. Water from natural sources including mineral cations can also be used, depending on the desired characteristic of the product.

Generally, the composition of the present invention comprises from about 10% to about 99%, preferably from about 50% to about 95%, more preferably from about 60% to about 90% by weight of the composition, of a total of aqueous carriers.

The pH of the present composition is preferably from about 4 to about 8, more preferably from about 5 to about 7. The suitable tacky skin treatment agents are particularly efficient in such pH range. Buffers and other pH adjusting agents can be included to achieve the desirable pH.

Carboxylic Acid/Carboxylate Copolymer

The compositions of the present invention comprise from about 0.01% to about 5%, preferably from about 0.04% to about 1% of a carboxylic acid/carboxylate copolymer.

Without being bound by theory, the carboxylic acid/carboxylate copolymer is believed to provide a shear thinning property to the present composition. What is meant by shear thinning property is that a yield point exists within a typical shear stress applicable by the hand on the skin, and that the viscosity of the composition beyond the yield point significantly decreases to the extent such decrease is noticeable by the consumer. In a preferred embodiment, the composition of the present composition, prior to application of shear, has a suitable viscosity, preferably from about 100 mPaes to about 6000 mPa·s, more preferably from about 500 mPa·s to about 4500 mPa·s, still preferably from about 700 mPa·s to about 4000 mPa·s. The viscosity herein can be suitably measured by Brookfield LV at 20 rpm at 25° C. using either spindle #4, 5, 6 or 7 depending on the viscosity and the characteristic of the composition. In a highly preferred embodiment, the viscosity beyond the yield point is similar to that of water. The shear thinning property provides a low viscous composition directly upon use, thereby providing the feel of applying water to the skin.

The carboxylic acid/carboxylate copolymer also keeps the composition relatively transparent and at a suitable viscosity without making the composition tacky or greasy upon use.

Additionally, the carboxylic acid/carboxylate copolymer is capable of dispersing and stabilizing the oily components in an aqueous environment, so that such components do not separate out. When first oily components are incorporated in the composition in addition to the amount encompassed in the first emulsion, the additional first oily components can be incorporated into the composition, by dispersing with the carboxylic acid/carboxylate copolymer. The additional first oily components are preferably silicone oils. The additional first oily components, dispersed with the carboxylic acid/carboxylate copolymer, can be included in the composition at a level of from about 0.05% to about 5%, preferably from about 0.5% to about 3% by weight of the composition.

The carboxylic acid/carboxylate copolymers herein are hydrophobically-modified cross-linked coplymers of carboxylic acid and alkyl carboxylate, and have an amphiphilic property. It is believed that, because of the alkyl group contained in the copolymer, the carboxylic acid/carboxylate copolymers do not make the composition undesirably sticky.

Suitable carboxylic acid/carboxylate copolymers herein are acrylic acid/alkyl acrylate copolymers having the following formula:

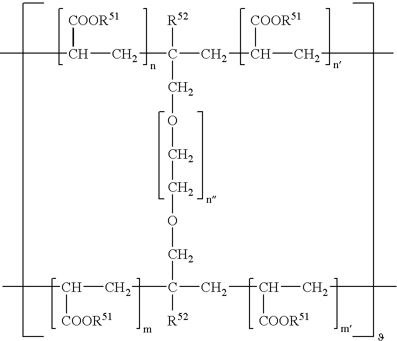

wherein R⁵¹, independently, is a hydrogen or an alkyl of 1 to 30 carbons wherein at least one of R⁵¹ is a hydrogen, R⁵² is as defined above, n, n′, m and m′ are integers in which n+n′+m+m′ is from about 40 to about 100, n” is an integer of from 1 to about 30, and l is defined so that the copolymer has a molecular weight of about 500,000 to about 3,000,000.

Commercially available carboxylic acid/carboxylate copolymers useful herein include: CTFA name Acrylates/C10-30 Alkyl Acrylate Crosspolymer having tradenames Pemulen TR-1, Pemulen TR-2, Carbopol 1342, Carbopol 1382, and Carbopol ETD 2020, all available from B. F. Goodrich Company.

Neutralizing agents may be included to neutralize the carboxylic acid/carboxylate copolymers herein. Nonlimiting examples of such neutralizing agents include sodium hydroxide, potssium hydroxide, ammonium hydroxide, monethanolamine, diethanolamine, triethanolamine, diisopropanolamine, aminomethylpropanol, tromethamine, tetrahydroxypropyl ethylenediamine, and mixtures thereof.

Additional Water Soluble Polymer

The compositions of the present invention may further comprise an additional water soluble polymer, preferably at a level of from about 0.01% to about 5%, more preferably from about 0.04% to about 1%. The additional water soluble polymers herein are water soluble or water miscible polymers, and are compatible with the carboxylic acid/carboxylate copolymers. Without being bound by theory, it is believed the controlled amount of additional water soluble polymers in the composition provides improved moisturization and smoothness to the skin without giving an undesirable tacky or sticky feeling.

In one preferred embodiment, the additional water soluble polymer is selected so that the composition of the present composition has a suitable viscosity of preferably from about 100 mPa·s to about 6000 mPa·s, as described above.

Additional water soluble polymers useful herein include anionic polymers and nonionic polymers. Useful herein include, for example, vinyl polymers such as cross linked acrylic acid polymers with the CTFA name Carbomer; cellulose derivatives and modified cellulose polymers such as methyl cellulose, ethyl cellulose, hydroxyethyl cellulose, hydroxyethyl ethylcellulose, hydroxypropyl methyl cellulose; polyvinylpyrrolidone; polyvinyl alcohol; gums such as guar gum, hydroxypropyl guar gum, and xanthan gum; microbiological polymers such as dextran and succinoglucan; starch-based polymers such as carboxymethyl starch and methylhydroxypropyl starch; acrylate polymers such as sodium polyacrylate, polyethylacrylate, and polyacrylamide. Polyalkylene glycols having a molecular weight of more than about 1000 are also useful herein. Additional water soluble polymers also useful herein include amphoteric polymers such as Polyquaternium 22, Polyquaternium 39, Polyquaternium 47, and octylacrylamine/acrylates/butylaminoethyl methacrylate copolymers.

Commercially available additional water soluble polymers highly useful herein include xanthan gum with tradename Keltrol series available from Kelco, Carbomers with tradenames Carbopol 934, Carbopol 940, Carbopol 950, Carbopol 980, and Carbopol 981, all available from B. F. Goodrich Company, acrylates/steareth-20 methacrylate copolymer with tradename ACRYSOL 22 available from Rohm and Hass, nonoxynyl hydroxyethylcellulose with tradename AMERCELL POLYMER HM-1500 available from Amerchol, methylcellulose with tradename BENECEL, hydroxyethyl cellulose with tradename NATROSOL, hydroxypropyl cellulose with tradename KLUCEL, cetyl hydroxyethyl cellulose with tradename POLYSURF 67, all supplied by Herculus, scleroglucan with tradename Clearogel SC11 available from Michel Mercier Products Inc. (NJ, USA), ethylene oxide and/or propylene oxide based polymers with tradenames CARBOWAX PEGs, POLYOX WASRs, and UCON FLUIDS, all supplied by Amerchol.

Water Soluble Humectant

The composition of the present invention comprises from about 1% to about 20%, preferably from about 5% to about 15% of a water soluble humectant. Water soluble humectants useful herein include polyhydric alcohols such as glycerin, sorbitol, propylene glycol, butylene glycol, hexylene glycol, ethoxylated glucose, 1,2-hexane diol, 1,2-pentane diol, hexanetriol, dipropylene glycol, erythritol, trehalose, diglycerin, xylitol, maltitol, maltose, glucose, fructose, sodium chondroitin sulfate, sodium hyaluronate, sodium adenosin phosphate, sodium lactate, pyrrolidone carbonate, glucosamine, cyclodextrin, and mixtures thereof.

Water soluble humectants useful herein include water soluble alkoxylated nonionic polymers such as polyethylene glycols and polypropylene glycols having a molecular weight of up to about 1000 such as those with CTFA names PEG-200, PEG-400, PEG-600, PEG-1000, and mixtures thereof.

Commercially available humectants highly useful herein include: butylene glycol with tradename 1,3-butylene glycol available from ASAHI DENKA Co. Ltd.; glycerin with tradenames CRODEROL GA7000 available from Croda Universal Ltd., PRECERIN series available from Unichema, and a same tradename as the chemical name available from NOF; propylene glycol with tradename LEXOL PG-865/855 available from Inolex, 1,2-PROPYLENE GLYCOL USP available from BASF; dipropylene glycol with the same tradename available from BASF; diglycerin with tradename DIGLYCEROL available from Solvay GmbH; polyethylene glycols with the tradename CARBOWAX series available from Union Carbide, and a mixture of glyceryl polymethacrylate, propylene glycol and PVM/MA copolymer with tradename Lubrajel Oil available from Guardian Lab.

Sebum Absorbing Agent

The composition of the present invention may contain from about 0.1% to about 10%, preferably from about 1% to about 5% of a sebum absorbing agent. Sebum absorbing agents useful herein include those which actually absorb the sebum discreted from the pores, and are compatible with the aqueous composition of the present invention. Components which are water soluble, water swellable, or have high emulsifying ability are not suitable herein, as they would no longer have sebum absorbing ability when formulated in the composition.

Preferable sebum absorbing agents herein include porous spherical cellulose powder, solid silicone elastomer powder, surface modified porous silica powder, porous nylon powder, porous acrylate copolymer, and mixtures thereof. The type and amount of sebum absorbing agents are selected according to the desired character of the product.

Commercially available porous spherical cellulose powders highly useful herein include the materials with tradename Celluflow series, such as Celluflow CO₂₅ available from Chisso Corp. Commercially available solid silicone elastomer powders highly useful herein include vinyl dimethicone/methicone silsesquioxane crosspolymer with tradenames KSP series available from ShinEtsu Chemical Co., Ltd., Tokyo Japan. Other commercially available sebum absorbing agents include porous acrylate copolymers with tradename Polytrap available from Dow Corning.

Tacky Skin Treatment Agent

The composition of the present invention may contain from about 0.5% to about 10%, preferably from about 1% to about 5% of a tacky skin treatment agent. Skin treatment agents useful herein are those which help repair and replenish the natural moisture barrier function of the epidermis, thereby providing skin benefits such as texture improvement. It is generally known that, while such agents provide useful benefits to the skin when used chronically, they also tend to provide negative skin feel upon use when applied by itself.

Tacky skin treatment agents useful herein are niacinamide, nicotinic acid and its esters, nicotinyl alcohol, panthenol, panthenyl ethyl ether, n-acetyl cysteine, n-acetyl-L-serine, phosphodiesterase inhibitors, trimethyl glycine, urea, gelatin, soluble collagen, royal jelly, tocopheryl nicotinate, and vitamin D3 and analogues or derivatives, and mixtures thereof. Niacinamide is particularly preferred in that, when used in a pharmaceutically effective amount, is capable of reducing or alleviating the intensity of chronical spots. Niacinamide is suitably incorporated in the composition by first dissolving in water. Panthenol is also particularly preferred in that, when used in an amount of at least about 1%, it provides texture improvement benefits. Niacinamide and panthenol are commercially available, for example, by Roche.

Sebum Supressing Plant Extract

The composition of the present invention may further comprise from about 0.001% to about 5%, more preferably from about 0.05% to about 1% of a sebum suppressing plant extract. The plant extracts useful herein are those which have an astringent type of effect for reducing the size of pores, or inhibition effect of 5-α-reductase, and are compatible with the aqueous form of the present composition, and preferably do not alter the transparent or translucent appearance of the present composition. Water soluble plant extracts are preferred. Useful plant extracts herein include clove (choji) extract, coix (yokuinin) extract, witch hazel (hamamerisu) extract, and mixtures thereof. Such plant extracts are available from Iwase.

UV Protecting Agent

The composition of the present invention may further comprise from about 1% to about 10%, more preferably from about 3% to about 9.5% of a UV protecting agent. UV protecting agents generally prevent excessive scaling and texture changes of the stratum corneum by exposure of ultraviolet light and may be added to the emulsion of the present invention. Suitable UV protecting agents may be organic or inorganic. Hydrophobic and water insoluble LUV protecting agents may be used at controlled levels.

A wide variety of conventional UV protecting agent are suitable for use herein. Preferred among those UV protecting agent which are useful in the emulsions are those selected from 2-ethylhexyl-p-methoxycinnamate (commercially available as PARSOL MCX), butylmethoxydibenzoyl-methane, 2-hydroxy-4-methoxybenzo-phenone, 2-phenylbenzimidazole-5-sulfonic acid, octyldimethyl-p-aminobenzoic acid, octocrylene, 2-ethylhexyl N,N-dimethyl-p-aminobenzoate, p-aminobenzoic acid, 2-phenylbenzimidazole-5-sulfonic acid, octocrylene, oxybenzone, homomenthyl salicylate, octyl salicylate, 4,4′-methoxy-t-butyldibenzoylmethane, 4-isopropyl dibenzoylmethane, 3-benzylidene camphor, 3-(4-methylbenzylidene) camphor, titanium dioxide such as MT-100 available from Tayca, zinc oxide, silica, iron oxide, Eusolex™ 6300, Octocrylene, Parsol 1789, and mixtures thereof.

Particularly useful herein are UV protecting agents such as those disclosed in U.S. Pat. No. 4,937,370, Sabatelli, issued Jun. 26, 1990, and U.S. Pat. No. 4,999,186, Sabatelli, issued Mar. 12, 1991. The UV protecting agents disclosed therein have, in a single molecular, two distinct chromophore moieties which exhibit different ultraviolet radiation absorption spectra. One of the chromophore moieties absorbs predominantly in the UVB radiation range and the other absorbs strongly in the UVA radiation range. These UV protecting agent provide higher efficacy, broader UV absorption, lower skin penetration and longer lasting efficacy relative to conventional UV protecting agent.

Exact amounts will vary depending upon the sunscreen chosen and the desired Sun Protection Factor (SPF). SPF is a commonly used measure of photoprotection of a sunscreen against erythema. See Federal Register, Vol. 43, No. 166, pp. 38206-38269, Aug. 25, 1978.

Whitening Agent

The composition of the present invention may further comprise from about 0.001% to about 10%, more preferably from about 0.1% to about 5% of a whitening agent. Whitening agents useful herein are those which are compatible with the aqueous form of the present composition. Water soluble whitening agents are preferred. The whitening agent useful herein refers to active ingredients that not only alter the appearance of the skin, but further improve hyperpigmentation as compared to pre-treatment.

Useful whitening agents useful herein include ascorbic acid compounds, azelaic acid, butyl hydroxy anisole, gallic acid and its derivatives, glycyrrhizinic acid, hydroquinoine, kojic acid, arbutin, mulberry extract, and mixtures thereof. Use of combinations of whitening agents is believed to be advantageous in that they may provide whitening benefit through different mechanisms.

Preferably, the ascorbic acid compound useful herein is an ascorbic acid salt or derivative thereof. Exemplary water soluble salt derivatives include, but are not limited to, L-ascorbic acid 2-glucoside, L-ascorbyl phosphate ester salts such as sodium L-ascorbyl phosphate, potassium L-ascorbyl phosphate, magnesium L-ascorbyl phosphate, calcium L-ascorbyl phosphate, aluminum L-ascorbyl phosphate. L-ascorbyl sulfate ester salts can also be used. Examples are sodium L-ascorbyl sulfate, potassium L-ascorbyl sulfate, magnesium L-ascorbyl sulfate, calcium L-ascorbyl sulfate and aluminum L-ascorbyl sulfate.

Additional Components

The compositions herein may further contain other additional components, which may be selected by the artisan according to the desired characteristics of the final product and which are suitable for rendering the compositions more cosmetically or aesthetically acceptable or to provide them with additional usage benefits. The components useful herein are conveniently categorized by a certain benefit or their postulated mode of action, however, a given category is not limiting of its use. Further, it is understood the one component may provide multiple benefits.

(i) Anti-Oxidants and Radical Scavengers

Anti-oxidants and radical scavengers are especially useful for providing protection against UV radiation which can cause increased scaling or texture changes in the stratum corneum and against other environmental agents which can cause skin damage. Preferred anti-oxidants/radical scavengers include, for example, tocopherol sorbate and other esters of tocopherol, more preferably tocopherol sorbate. For example, the use of tocopherol sorbate in topical emulsions and applicable to the present invention is described in U.S. Pat. No. 4,847,071, Bissett et al, issued Jul. 11, 1989.

(ii) Anti-Inflammatory Agents

Anti-inflammatory agents enhance the skin appearance benefits, by for example, contribution of uniformity and acceptable skin tone and/or color. Preferably, the anti-inflammatory agent includes a steroidal anti-inflammatory agent and a non-steroidal anti-inflammatory agent. Preferred steroidal anti-inflammatory for use is hydrocortisone. So-called “natural” anti-inflammatory agents are also useful. For example, alpha bisabolol, aloe vera, Manjistha (extracted from plants in the genus Rubia, particularly Rubia Cordifolia), and Guggal (extracted from plants in the genus Commiphora, particularly Commiphora Mukul), kola extract, chamomile, and sea whip extract, may be used.

(iii) Antimicrobial Agent

As used, “antimicrobial agents” means a compound capable of destroying microbes, preventing the development of microbes or preventing the pathogenic action of microbes. Antimicrobal agents are useful, for example, in controlling acne. Preferred antimicrobial agents useful in the present invention are benzoyl peroxide, erythromycin, tetracycline, clindamycin, azelaic acid, sulfur resorcinol, phenoxyethanol, and Irgasan™ DP 300 (Ciba Geigy Corp., U.S.A.). A safe and effective amount of an antimicrobial agent may be added to emulsions of the present invention, preferably from about 0.001% to about 10%, more preferably from about 0.01% to about 5%, still more preferably from about 0.05% to about 2%.

(iv) Chelators

As used herein, “chelator” refers to a compound that reacts for removing a metal ion from a system by forming a complex so that the metal ion cannot readily participate in or catalyze chemical reactions. The inclusion of a chelator is especially useful for providing protection against UV radiation which can contribute to excessive scaling or skin texture changes and against other environmental agents which can cause skin damage. Exemplary chelators that are useful herein are disclosed in U.S. Pat. No. 5,487,884, Bissett et al, issued Jan. 30, 1996; PCT application 91/16035 and 91/16034, Bush et al, published Oct. 31, 1995. Preferred chelators are furildioxime and derivatives thereof.

(v) Other Components

In addition to the above described components, the composition of the present invention may further include preservatives and preservative enhancers such as water-soluble or solubilizable preservatives including Germall 115, methyl, ethyl, propyl and butyl esters of hydroxybenzoic acid, benzyl alcohol, imidazolidinyl urea, EDTA and its salts, Bronopol (2-bromo-2-nitropropane-1,3-diol) and phenoxypropanol; antifoaming agents; binders; biological additives; bulking agents; coloring agents; essential oils and solubilizers thereof; other natural extracts; compounds which stimulate collagen production; yeast fermented filtrates, and others.

Method of Preparation

Preferably, the composition of the present invention is prepared by a method of preparation comprising the steps of:

(a) preparing a first mixture comprising the steps of:

(a1) mixing the first oily component with the first surfactant system and the first aqueous carrier at a temperature of about 70-80° C. to form a first emulsion; the amount of the first aqueous carrier being from about 0.5 to about 10 times the total weight of the first oily component and the first surfactant system;

(a2) cooling the mixture of (a1) to about 50° C.;

(a3) adding the mixture of (a2) to the third aqueous carrier, the temperature of the third aqueous carrier being cold enough to immediately cool the obtained mixture to a temperature of no higher than 40° C.;

(b) preparing a second mixture comprising the step of:

(b1) mixing the second oily component with the second surfactant system and the second aqueous carrier at a temperature of about 20-40° C. to form the second emulsion; the amount of the second aqueous carrier being from about 0.5 to about 20 times the total weight of the second oily component and the second surfactant system;

(c) mixing the first mixture and the second mixture.

While not essential to the present method, a polyol carrier, a carboxylic acid/carboxylate copolymer, and a water soluble humectant are typically added. The components used in the present method are the same as those described above.

A first mixture is prepared with the first surfactant, the first oily component, the first aqueous carrier, and the third aqueous carrier. In step (a1), the first oily component, the first surfactant system, and the first aqueous carrier are mixed together in an anchor mixer at a temperature of about 70-80° C. to form the first emulsion. Preferably, the first surfactant system is first mixed with the first oily component, and such mixture is added to the first aqueous carrier. The amount of the first aqueous carrier used in step (a1) is preferably from about 0.5 to about 10 times, more preferably from about 1 to about 7 times the total weight of the first oily component and the first surfactant system. In step (a2), the mixture of step (a1) is cooled to 50° C., preferably within a period of about 20 minutes. It has been discovered that such cooling allows the mixture to become transparent. In step (a3), the mixture of step (a2) is further cooled by mixing with the third aqueous carrier. The third aqueous carrier to be mixed with the mixture of step (a2) has a temperature cold enough that the obtained mixture is immediately cooled to no higher than 40° C., preferably about 30° C. Without being bound by theory, it is believed this second cooling process provides a stable transparent mixture with as small amount of surfactant as possible.

Separately, a second emulsion is prepared with the second surfactant, the second oily component, and the second aqueous carrier. In step (b1), the second oily component, the second surfactant system, and the second aqueous carrier are mixed together in an anchor mixer at a temperature of about 20-40° C. to form the second emulsion. Preferably, the second surfactant system is first mixed with the second oily component, and such mixture is added to the second aqueous carrier. The amount of the second aqueous carrier used in step (b1) is preferably from about 0.5 to about 20 times, more preferably from about 1 to about 10 times the total weight of the second oily component and second surfactant system. Polyol carriers are preferably added to the second emulsion. Preferably, the polyol carriers are mixed with the second surfactant system prior that the second surfactant system is mixed with the second oily component.

Finally, the first mixture and the second mixture are mixed until homogeneous (Step c). Mixing at this step is preferably conducted by an anchor mixer at a relatively low rotation speed, preferably from about 50 rpm to about 100 rpm. This step can be carried out at room temperature.

In the composition of the present invention, carboxylic acid/carboxylate copolymer and water-soluble humectant are preferably added. Preferably, the carboxylic acid/carboxylate copolymer, and if present, additional water soluble polymers are dispersed in a fourth aqueous carrier at a temperature of about 70-80° C. until homogeneous. When these polymers are dispersed, a high speed agitator may be used and the rotation speed is controlled to no more than about 5000 rpm to avoid destruction of the polymer structures. When neutralizing agents are present, they are added subsequent to this dispersion. Preferably water soluble humectant is added to this polymeric mixture. The remaining components, if present, such as sebum absorbing agents, tacky skin treatment agents, sebum suppressing plant extracts, UV absorbing agents, whitening agents, and preservatives may be added to this polymeric mixture. This polymeric mixture, when included, is added to the mixture of step (c).

In the composition of the present invention, when carboxylic acid/carboxylate copolymer is contained, first oily components such as silicone oils may be contained in the present composition by dispersing with this carboxylic acid/carboxylate copolymer, in addition to the amount encompassed in the first emulsion. In this case, the additional first oily components are preferably added to the above polymeric mixture.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

Compositions

| Phase | | Ex. 1 | Ex. 2 | Ex. 3 | Ex. 4 | Ex. 5 | Ex. 6 |
|---|---|---|---|---|---|---|---|
| 01 | Ceteareth-12 | 0.013 | 0.01 | 0.02 | 0.04 | 0.02 | 0.013 |
| 01 | Ceteareth-20 | 0.05 | 0.05 | 0.13 | 0.14 | 0.054 | 0.05 |
| 01 | Ceteareth-30 | 0.09 | 0.1 | 0.15 | 0.24 | 0.097 | 0.09 |
| 01 | Polyglyceryl-3 Diisostearate | 0.14 | 0.1 | 0.32 | 0.39 | 0.156 | 0.14 |
| 01 | PEG-20 Hydrogenated Castor Oil | 0.04 | 0.04 | 0.15 | 0.12 | 0.048 | 0.04 |
| 01 | Cetyl 2-ethylhexanoate | 0.025 | 0.03 | 0.23 | 0.07 | 0.028 | 0.025 |
| 01 | Meadowfoam Seed Oil *1 | 0.05 | 0.05 | 0.15 | 0.15 | 0.06 | 0.05 |
| 01 | Isohexadecane | — | — | — | 0.1 | 0.1 | — |
| 01 | Isononyl Isononanoate | — | — | — | — | — | 0.1 |
| 01 | Benzyl alcohol | 0.3 | 0.3 | 0.3 | — | 0.12 | 0.2 |
| 02 | De ionized Water | 1.6 | 1.6 | 4.3 | 4.3 | 1.72 | 1.6 |
| 03 | De ionized Water | 30.0 | 30.0 | 30.0 | 30.0 | 30.0 | 30.0 |
| 03 | Sodium Benzoate | 0.07 | 0.07 | 0.07 | 0.07 | 0.07 | 0.07 |
| 03 | EDTA 2-Na | 0.04 | 0.04 | 0.04 | 0.04 | 0.04 | 0.04 |
| 03 | Glycerin | 0.72 | 2.0 | — | 2.0 | — | 2.0 |
| 03 | Glyceryl Polymethacrylate & Propylene | 1.5 | — | 1.5 | 2.0 | — | — |
| | Glycol & PVM/MA copolymer *2 | | | | | | |
| 03 | 1,3-Butylene glycol | 2.3 | 2.3 | 2.3 | 2.3 | 2.3 | 2.3 |
| 03 | Methyl Paraben | 0.07 | 0.07 | 0.07 | 0.07 | 0.07 | 0.07 |
| 04 | Menthol | 0.01 | — | 0.01 | 0.05 | 0.05 | — |
| 04 | Perfume *3 | 0.05 | 0.05 | 0.06 | — | 0.05 | — |
| 04 | Tea tree oil *4 | — | — | — | — | — | 0.1 |
| 04 | PEG-40 Hydrogenated Castor Oil | 0.4 | 0.5 | — | 0.2 | 0.4 | 0.4 |
| 04 | PEG-20 Hydrogenated Castor Oil | 0.2 | — | — | — | — | 0.2 |
| 04 | Polysorbate-20 | — | — | — | 0.3 | 0.2 | — |
| 04 | Polyglyceryl-6 laurate | — | — | 0.6 | — | — | — |
| 04 | 1,3-Butylene Glycol | 0.6 | 0.6 | 0.6 | 0.6 | 0.6 | 0.6 |
| 05 | De ionized water | 2.0 | 2.0 | 2.0 | 2.0 | 2.0 | 2.0 |
| 06 | Acrylic acid/alkyl acrylate copolymer 1 *5 | — | — | — | — | 0.23 | 0.2 |
| 06 | Acrylic acid/alkyl acrylate copolymer 2 *6 | 0.25 | 0.25 | 0.25 | 0.3 | — | — |
| 06 | De ionized Water | balance to 100% | | | | | |
| 07 | De ionized water | 5.0 | 5.0 | 5.0 | 5.0 | 5.0 | 5.0 |
| 08 | Sodium Hydroxide | 0.07 | 0.1 | 0.1 | 0.85 | 0.2 | 0.06 |
| 08 | De ionized water | — | — | — | 0.12 | 0.15 | 0.1 |
| 09 | Dimethicone/Dimethiconol *7 | 1.5 | — | 2.0 | — | 1.0 | — |
| 09 | Cyclomethicone/Dimethiconol *8 | — | 1.5 | — | 1.5 | — | 1.0 |
| 10 | De ionized water | 20.0 | 20.0 | 20.0 | 20.0 | 20.0 | 20.0 |
| 10 | Methyl paraben | 0.1 | 0.05 | 0.1 | 0.05 | 0.07 | 0.07 |
| 10 | 1,3-butylene glycol | 4.0 | 2.0 | 4.0 | 3.0 | 3.0 | 4.0 |
| 10 | Xanthan gum *9 | 0.04 | 0.04 | 0.04 | 0.04 | — | — |
| 10 | Carbomer *10 | — | — | — | — | 0.2 | — |
| 10 | Sodium Hyaluronate *11 | 0.05 | — | — | — | 0.02 | 0.02 |
| 11 | D-panthenol *12 | — | 1.0 | 1.0 | 1.0 | 2.0 | 1.0 |
| 11 | Niacinamide *13 | — | 2.0 | 2.0 | 2.0 | 2.0 | 2.0 |
| 11 | Zizyphus Jujuba Fruit Extract *14 | 0.1 | — | 0.15 | 0.1 | — | 0.15 |
| 11 | Witch Hazel Extract *15 | — | — | — | 0.1 | — | — |
| 11 | Clove Extract *16 | — | — | 0.1 | — | — | — |
| 11 | Magnesium Ascorbyl Glucoside | — | — | — | — | 2.0 | — |
| 11 | Benzophenone-1 | — | — | — | — | — | 2.0 |
| 11 | Titanium Dioxide *17 | — | — | — | — | — | 1.0 |
| 11 | Cellulose Powder *18 | — | — | — | — | 3.0 | — |
| 11 | Vinyl Dimethicone/methicone | — | — | — | — | — | 5.0 |
| | Silsesquioxane Crosspolymer *19 | | | | | | |
| 11 | De ionized water | 10 | 10 | 10 | 10 | 10 | 10 |

| Phase | | Ex. 7 | Ex. 8 |
|---|---|---|---|
| 01 | Ceteareth-12 | 0.013 | 0.01 |
| 01 | Ceteareth-20 | 0.05 | 0.05 |
| 01 | Ceteareth-30 | 0.09 | 0.1 |
| 01 | Polyglyceryl-3 Diisostearate | 0.14 | 0.1 |
| 01 | PEG-20 Hydrogenated Castor Oil | 0.04 | 0.04 |
| 01 | Cetyl 2-ethylhexanoate | 0.025 | 0.03 |
| 01 | Meadowfoam Seed Oil *1 | 0.05 | 0.05 |
| 01 | Benzyl alcohol | 0.3 | 0.3 |
| 02 | De ionized Water | 1.6 | 1.6 |
| 03 | De ionized Water | 30.0 | 30.0 |
| 03 | Sodium Benzoate | 0.07 | 0.07 |
| 03 | EDTA 2-Na | 0.04 | 0.04 |
| 03 | Glycerin | 0.72 | 2.0 |
| 03 | Glyceryl Polymethacrylate & Propylene | 1.5 | — |
| | Glycol & PVM/MA copolymer *2 | | |
| 03 | 1,3-Butylene glycol | 2.3 | 2.3 |
| 03 | Methyl Paraben | 0.07 | 0.07 |
| 04 | Menthol | 0.01 | — |
| 04 | Perfume *3 | 0.05 | 0.05 |
| 04 | Sodium Dilaureth-10 Phosphate | 0.45 | — |
| 04 | Glycereth-25 PCA Ispstearate | — | 0.45 |
| 04 | 1,3-Butylene Glycol | 0.6 | 0.6 |
| 05 | De ionized water | 2.0 | 2.0 |
| 06 | Acrylic acid/alkyl acrylate copolymer 2 *6 | 0.25 | 0.25 |

| | | |
|---|---|---|
| 06 | De ionized Water | balance to |
| | | 100% |

| | | | |
|---|---|---|---|
| 07 | De ionized water | 5.0 | 5.0 |
| 08 | Sodium Hydroxide | 0.07 | 0.1 |
| 09 | Dimethicone/Dimethiconol *7 | 1.5 | — |
| 09 | Cyclomethicone/Dimethiconol *8 | — | 1.5 |
| 10 | De ionized water | 20.0 | 20.0 |
| 10 | Methyl paraben | 0.1 | 0.05 |
| 10 | 1,3-butylene glycol | 4.0 | 2.0 |
| 10 | Xanthan gum *9 | 0.04 | 0.04 |
| 10 | Sodium Hyaluronate *11 | 0.05 | — |
| 11 | D-panthenol *12 | — | 1.0 |
| 11 | Niacinamide *13 | — | 2.0 |
| 11 | Zizyphus Jujuba Fruit Extract *14 | 0.1 | — |
| 11 | De ionized water | 10 | 10 |

Definitions of Components
*1 Meadowfoam Seed Oil: Cropure MDF available from Croda
*2 Glyceryl Polymethacrylate & Propylene Glycol & PVM/MA copolymer: Lubrajel Oil available from Guardian Lab
*3 Perfume: containing limonene
*4 Tea tree oil: containing terpinene-4-ol and γ-terpinene available from Southern Cross Botanicals.
*5 Acrylic acid/alkyl acrylate copolymer 1: PEMULEN TR-1 available from B. F. Goodrich
*6 Acrylic acid/alkyl acrylate copolymer 2: PEMULEN TR-2 available from B. F. Goodrich
*7 Dimethicone/Dimethiconol: DCQ2-1403 available from Dow Corning
*8 Cyclomethicone/Dimethiconol: DCQ2-1401 available from Dow Corning
*9 Xanthan gum: Keltrol T available from Kelco
*10 Carbomer: Carbopol 981 available from B. F. Goodrich
*11 Sodium Hyaluronate: available from Chisso corp.
*12 Panthenol: available from Roche
*13 Niacinamide: available from Roche
*14 Zizyphus Jujuba Fruit Extract: Available from Ichimaru Pharcos
*15 Witch Hazel Extract: Hamamerisu Liquid available from Iwase
*16 Clove Extract: Choji Extract BG available from Iwase
*17 Titanium Dioxide: Titanium Dioxide MT-100 available from Tayc
*18 Cellulose Powder: Celluflow C-25 available from Chisso corp.
*19 Vinyl dimethicone/methicone Silsesqueioxane Crosspolymer: KSP-100 available from Shinetsu Chemical

Method of Preparation

(a1) Heat Phase 01 to about 80° C. Heat Phase O₂ to about 78° C. Emulsify by adding Phase 01 to Phase O₂ and mixing.

(a2) Cool the mixture of (a1) to about 50° C. within a period of about 10 to about 20 minutes.

(a3) Add the mixture of (a2) to Phase 03, Phase 03 being at room temperature.

(b1) Emulsify by adding Phase 04 to Phase 05 at about 25° C.

(c) Mix the mixture of step (a3) and the product of step (b1).

(d1) When included, heat Phase 06 to about 80° C., and mix Phase 06 with high speed agitator at no more than 5000 rpm until homogenous.

(d2) Add Phase 06 to Phase 07 of about 80° C., and cool the mixture to about 45° C.

(d3) Add Phase 09 to the mixture of (d2).

(d4) Add Phase 08 to the mixture of (d4).

(d5) Heat Phase 10 to about 80° C. and mix Phase 10 with high speed agitator at no more than 5000 rpm until homogenous. Add Phase 10 to the mixture of (d4).

(d6) Mix Phase 11 with high speed agitator at no more than 5000 rpm until homogenous. Add Phase 11 to the mixture of (d5). Finally mix the mixture of step (c) and the mixture of step (d6).

The embodiments disclosed and represented by the previous examples have many advantages. For example, Examples 1 through 8 are particularly useful for providing clear lotions for use on the facial skin. When used on the facial skin, the compositions of Examples 1 through 8 can provide moisturizing benefit, smoothness and softness to the skin without leaving a tacky and/or greasy feel to the skin.

In Examples 1 through 8, the base compositions comprising Phases 01, 02, 03, 04, and 05 have a transparent appearance, and an absorbance of no more than about 2 at a wave length of 340 nm. The compositions of Examples 1 through 8 have translucent appearance. The compositions of Examples 1 through 5, 7, and 8 can provide an aesthetic benefit due to perfume and/or menthol. The composition of Example 6 can provide an additional conditioning benefit due to tea tree oil.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A skin care composition comprising:
(a) a first emulsion comprising a first surfactant system, a first oily component, and a first aqueous carrier; wherein the first surfactant system comprises two or more nonionic surfactants selected from the group consisting of polyoxyalkylene alkyl ethers having the C12-18 of alkyl substitute, polyoxyalkylene hydrogenated caster oils, and a linear or branched, mono-or tri-alkyl glycerides; wherein the first oily component is selected from the group consisting of emollients, silicone oils, and mixtures thereof; and wherein the weight ratio of the first surfactant system to the first oily component is from about 5:1 to about 1:1;
(b) a second emulsion comprising a second surfactant system, a second oily component, and a second aqueous carrier; wherein the second surfactant system comprises one or more nonionic surfactants selected from the group consisting of polyoxyalkylene hydrogenated caster oils, polyglycerin alkyl esters having the C10-20 of alkylsubstitute, polysorbates, polyoxyethylene sterols, polyoxyethylene hydrogenated sterols, hydroxylated lecithin, polyoxyethylene alkyl ether phosphates and salts thereof, and glycereth-25 PCA isostearate; wherein the second oily component is selected from the group consisting of perfumes, hydrophobic plant extracts containing terpene compounds, hydrophobic vitamins, and mixtures thereof; and wherein the weight ratio of the second surfactant system to the second oily component is from about 20:1 to about 3:1; and
(c) a third aqueous carrier.

2. The skin care composition of claim 1 wherein a base composition consisting essentially of components (a) through (c) has an absorbance of no more than about 2 at a wave length of 340 nm.

3. The skin care composition of claim 1 wherein the first oily component comprises an emollient.

4. The skin care composition of claim 1 wherein the microemulsion has a mean particle size of less than about 100 nm.

5. The skin care composition of claim 1, wherein the first surfactant system has an HLB of 10 or more.

6. The skin care composition of claim 1, wherein the first system comprises at least one nonionic surfactant having an HLB of 10 or more and at least one nonionic surfactant having an HLB of below 10.

7. The skin care composition of claim 1 wherein the second surfactant system has an HLB of 8 or more.

8. The skin care composition of claim 1 wherein the second surfactant system consists essentially of nonionic surfactants having an HLB of 8 or more.

9. The skin care composition of claim 1 wherein the second emulsion further comprises a polyol carrier.

10. The skin care composition of claim 1 further comprising a carboxylic acid/carboxylate copolymer.

11. The skin care composition of claim 1 further comprising an additional water soluble polymer.

12. The skin care composition of claim 1 further comprising an additional water soluble humectant.

13. A method of preparation of a skin care composition of claim 1 comprising the steps of:
(a) preparing a first mixture comprising the steps of:
(a1) mixing the first oily component with the first surfactant system and the first aqueous carrier at a temperature of about 70-80° C. to form the first emulsion; the amount of the first aqueous carrier being from about 0.5 to about 10 times the total weight of the first oily component and the first surfactant system;
(a2) cooling the mixture of (a1) to about 50° C.;
(a3) adding the mixture of (a2) to the third aqueous carrier, the temperature of the third aqueous carrier being cold enough to immediately cool the obtained mixture to a temperature of no higher than 40° C.;
(b) preparing a second mixture comprising the step of:
(b1) mixing the second oily component with the second surfactant system and the second aqueous carrier at a temperature of about 20-40° C. to form the second emulsion; the amount of the second aqueous carrier being from about 0.5 to about 20 times the total weight of the second oily component and the second surfactant system;
(c) mixing the first mixture and the second mixture.
